(19) United States
(12) Patent Application Publication
(10) Pub. No.: US 2012/0123428 A1
(43) Pub. Date: May 17, 2012

(54) Repositioning Forceps With A Drilling Aid

(76) Inventor: Sascha Berberich, Tuttlingen (DE)

(21) Appl. No.: 13/293,307

(22) Filed: Nov. 10, 2011

(30) Foreign Application Priority Data
Nov. 10, 2010 (DE) 10 2010 051 919.7

(51) Int. Cl. A61B 17/90

(52) U.S. Cl. 606/96

(57) ABSTRACT

A repositioning forceps serves for holding two bone parts together which bone parts are to be connected. Two scissors-like forceps halves are connected via a hinge. Each of said two forceps halves has a distal section having a laterally outwardly curved branch and a proximal section having a grip part. The laterally outwardly curved branch has distal ends directed towards each other in a closed position of the reposition forceps. A drilling aid is mounted at one of said curved branches. Said drilling aid is rotatable at least about a rotation axis extending perpendicular to said forceps plane. Said drilling aid has a drilling tool guide extending along a guide axis extending along said branch.

### BACKGROUND OF THE INVENTION

The invention relates to repositioning forceps for holding two bone parts that are to be connected, with two forceps halves connected like scissors via a hinge and pivotable in a forceps plane, wherein each forceps half has, in the distal direction from the hinge, a laterally outwardly curved branch and, in the proximal direction, a grip part, and wherein a drilling aid is mounted movably on one branch and has a guide for a drilling tool.

A forceps for clamping two bone parts is known from U.S. Pat. No. 2,181,746.

Forceps for repositioning two bone parts are used in particular in emergency surgery where bone fragments that have splintered off from a bone are intended to be reattached to the bone. This attachment is done via screws or fixing wires.

Repositioning forceps are used to position the splintered-off bone fragment exactly on the bone.

U.S. Pat. No. 2,181,746 shows a bone clamp that holds two bone fragments together.

A relatively elongate drilling aid in the form of a sleeve extends laterally from one branch. A guide channel for a drilling tool is present centrally in the sleeve. In a starting position, the sleeve-like drilling aid extends laterally and approximately at right angles away from the branch on which it is mounted. The longitudinal axis of the drilling guide extends approximately in the forceps plane. Longitudinal slits are formed lengthwise in both branches such that, in the starting position of the drilling aid, it is possible to drill through the longitudinal slits of the branches. The drilling sleeve is mounted on a dome-like guide, such that the drilling aid, seen in the longitudinal direction of the branch, can be tilted sideways. However, the drilling tool has to be guided at least through the longitudinal slit in the branch on which the drilling tool is mounted.

A disadvantage of this device is that the drilling aid protrudes far sideways and therefore has a very bulky structure.

In addition, the degrees of freedom in the orientation of the drilling tool are limited by the fact that it is always necessary to drill through the central opening of the branch on which the drilling aid is mounted.

This greatly restricts the freedom of the operating surgeon in terms of where he wishes to set the bore. In the case of bone clamps that are intended to hold two bone parts in an osteotomy procedure, this is not particularly critical, since the two bone parts to be connected were separated beforehand with precisely defined cuts, for example in order to correct defective positions or excessive lengths. Therefore, these bone parts can be easily fixed via the defined cut.

In the case of bone splinters of complex shape, the operating surgeon requires considerable degrees of freedom for the drilling aid so as to be able to orient the drill channels to a position that provides sufficient bone substance for the fixing procedure. This freedom must be provided in particular if the procedure is to be performed subcutaneously.

It is therefore an object of the present invention is therefore to make available repositioning forceps that have a compact structure and are easy to use and that provide the operating surgeon with a high degree of freedom as regards setting the bore in the bone parts that are to be connected.

### SUMMARY OF THE INVENTION

According to the invention, the object is achieved by a repositioning forceps comprising two scissors-like forceps halves, a hinge connecting said two forceps halves like scissors, said two scissors-like forceps halves connected via said hinge extend and are movable in a forceps plane, each of said two forceps halves has a distal section distally from said hinge and a proximal section proximally from said hinge, each of said two forceps halves has a grip part at said proximal section, each of said two forceps halves has a laterally outwardly curved branch in said distal section, said curved branches having distal ends directed towards each other in a closed position of said reposition forceps, a drilling aid mounted at one of said curved branches, said drilling aid being rotatable at least about a rotation axis extending perpendicular to said forceps plane, said drilling aid comprising a drilling tool guide having a guide axis extending, in a particular rotation position of said rotatable drilling aid along said branch having mounted said drilling aid, but can deviate from an extension along said branch when said drilling aid was turned about said rotation axis.

In order to hold the bone parts, the branches are moved towards these and position the two bone parts that are to be connected between the distal ends of the branches. The orientation of the guide axis of the drilling aid in a rotation position along the branch now makes it possible to drill precisely in the direction of the bone fragments held between the distal ends. This procedure can also take place subcutaneously, since the physician of course knows that the bone fragments that are to be connected are held between the tips of the forceps. A pivoting of the drilling aid about an axis perpendicular to the pivoting plane makes it possible to mount the drilling aid onto a branch, but at a slight distance from the forceps plane, such that, in all rotation positions of the drilling aid, the bone fragments can be targeted and a branch is not hit, with the result that no arrangements have to be made to ensure that a drilling can done through this branch.

It is also possible for the entire drilling aid to be made very compact, since there are no guide channels protruding at right angles from the branches. A drilling tool inserted into the drilling aid can extend in a plane close and approximately parallel to the forceps plane. But due to the rotatability about an axis perpendicular to that plane, the drilling tool can target different targets at the bone parts held by the forceps.

This greatly facilitates the handling of the drilling aid and provides the operating surgeon with numerous degrees of freedom in terms of the orientation of the drilling aid.

In an embodiment of the invention, the drilling aid is designed as a block through which said drilling tool guide extends as at least one guide channel.

This measure has the advantage, in terms of handling, that a very compact drilling aid can be produced that can be very easily gripped by the operating surgeon and oriented in the desired drilling direction. To do so, the surgeon simply has to take hold of the block and turn it accordingly about the rotation axis so as to acquire the desired orientation.

In another embodiment of the invention, at least two guide channels are formed in the block.

This measure has the advantage that at least two drill channels can be produced with one orientation of the drilling aid, namely by virtue of the fact that several drilling tools can be guided through the at least two or more guide channels, or the same drilling tool can be guided through little by little, in order to produce several drill channels for fixing screws or fixing wires.

In another embodiment of the invention, the guide axes of the several guide channels extend at different heights and/or non-parallel to each other, but are nonetheless oriented with respect to an area between the two branches.

By means of this embodiment, two drill channels that are favourable for the fixing procedure and are oriented in different ways can be produced independently of each other, it being ensured that the drill channels do not meet, even in the case of a non-parallel orientation.

This allows the operating surgeon, in one orientation of the drilling aid, to gradually produce two drill channels that permit an optimal orientation for placement of the fixing screws or fixing wires, such that this procedure can be performed very quickly. Depending on the rotation position and orientation, the drill channels can intersect, but without meeting each other, or diverge.

In another embodiment, the block is connected to the branch by a pin extending in the pivot axis.

This measure has the advantage that the compact structure of the drilling aid as a block can be connected to the branch via a single pin. In this way, few structural parts are needed for the connection between drilling aid and branch, the latter also having few bacterial niches, such that a device of this kind can also be easily cleaned and disinfected.

In another embodiment of the invention, the extent of the rotation movement about the rotation axis is limited.

The aforementioned simple connection between the branch and the block via a pin now makes it possible in principle to pivot the block through 360° about the rotation axis. This may be desirable or favourable if the block is to be used such that the drilling tool can be pushed in through the block from two opposite directions, particularly if the guide channels are not parallel or run at different heights. Thus, for example, in one orientation of the block, it is possible to form drill channels directed towards each other and then, in the position rotated through 180°, to form suitably diverging channels through the former channels.

If however, as has already been mentioned, the channels are already formed in variable orientations in the drilling aid, it may be sufficient to provide only a certain degree of rotation, for example through a defined angle to the left and to the right about the pin.

For this purpose, in another embodiment, abutments are provided on the block on both sides of the branch and limit a rotation movement about the rotation axis.

By structurally simple measures, these abutments provide the aforementioned limiting of the rotation.

Such abutments can be designed as projections or protruding studs or the like and therefore as uncomplicated structural parts that are easy to produce. This not only facilitates simple production and handling, but also the cleaning and disinfecting of such repositioning forceps.

In another embodiment of the invention, the drilling aid is additionally designed to be rotatable about a longitudinal axis of the branch on which it is mounted.

This measure has the advantage that, by means of the rotation about the longitudinal axis, the physician can be afforded further degrees of freedom in producing the bores.

The drilling aid is mounted in a slightly laterally offset position on the branch. If the forceps are used to reattach a splintered bone fragment to a bone, the branches can be applied in such a way that they grip the broken-off bone fragment more or less centrally. In one position of the drilling aid, one or more bores can then be drilled on this side of the branch. Thereafter, the drilling aid can be rotated, for example through 180°, about the longitudinal axis of the branch, such that the bone fragment can then be provided with corresponding bores at a diametrically opposite location.

In the case of bone fragments having quite large surface areas, bores can now be produced in several different rotation positions about the longitudinal axis of the branch.

For storage or transport, the drilling aid can then be rotated into a position in which, with the forceps closed, it comes to lie inside of the two branches, which is favourable for stowage, packing and shipment.

In another embodiment of the invention, the drilling aid is designed to be movable along the branch on which it is mounted.

This measure permits additional fine adjustment, by virtue of the fact that the drilling aid can additionally have the degree of freedom of longitudinal movement along the branch.

In another embodiment of the invention, the two branches of the forceps halves are designed at the distal end as needle-like spikes.

This measure has the advantage that the repositioning can be performed subcutaneously, for example by the spikes penetrating the skin in order to reposition the bone fragments lying beneath the latter. This facilitates the surgical procedure, since the operating site does not have to be completely opened.

In another embodiment of the invention, the branches, seen from the central longitudinal axis of the repositioning forceps, have laterally outwardly curved portions, ending distally as needle-like spikes directed towards each other when the repositioning forceps is closed.

This measure has the advantage that the two bone fragments that are to be connected are held at a point between the needle-like spikes, and the outwardly curved portions ensure that sufficient space is present around this site to perform the necessary manoeuvres on the drilling aid.

This is very advantageous in particular if a relatively small splintered bone fragment is to be secured to a bone.

In another embodiment of the invention, the drilling aid is arranged near a straight needle-like spike.

This measure has the advantage that the proximity gives the operating surgeon a very good feel for how the bore produced by the drilling aid is oriented. The operating surgeon sees the orientation of the spikes, even when these are pushed through tissue, and he then knows how he has to orient the drilling aid in order to ensure that the bore exactly meets the bone fragments that are to be connected, even though he cannot see the bone fragments directly.

This greatly facilitates handling.

In another embodiment of the invention, a holding device is present between the grip parts of the forceps halves and holds the grip parts and therefore also the branches in defined positions of pivoting.

This measure known per se has the advantage that, in combination with the drilling aid according to the invention, the repositioning procedure can be performed particularly simply and safely. First, by pivoting of the two forceps halves, the two bone fragments that are to be connected can be brought into a desired position relative to each other, after which the holding device holds the forceps halves in these positions. The physician can then concentrate fully on the orientation of the drilling aid and produce the drill channels for the fixing screws or fixing wires.

It will be appreciated that the aforementioned features and those still to be explained below can be used not only in the respectively cited combination, but also in other combinations or singly, without departing from the scope of the present invention.

### BRIEF DESCRIPTION OF THE DRAWINGS

Illustrative embodiments of the invention are explained in more detail in the following description and are depicted in the drawing, in which:

FIG. 1 shows a plan view of a first illustrative embodiment of repositioning forceps with a drilling aid, which is designed to be movable only about one degree of freedom,

FIG. 2 shows a greatly enlarged plan view of part of the forceps from FIG. 1, specifically from the opposite side,

FIGS. 3A to 3C each show a greatly enlarged detail of the branch shown on the left-hand side in FIG. 2, with three different rotation positions of the drilling aid, namely a central position, a position of maximum rotation in one direction and the position of maximum rotation in the opposite direction, these being limited by corresponding abutments,

FIG. 4 shows a front view of the branch shown on the right-hand side in FIG. 1 with the drilling aid, specifically looking exactly towards the tip of the spike,

FIG. 5 shows a corresponding view from the opposite side, that is to say from the rear or in the direction from proximal to distal,

FIG. 6 shows a greatly enlarged view of part of the repositioning forceps from FIG. 1 in practical use,

FIG. 7a shows a second illustrative embodiment of repositioning forceps with a drilling aid, wherein this drilling aid is additionally pivotable about the longitudinal axis of the branch on which it is mounted,

FIG. 7b shows a position comparable to the view in FIG. 7a, wherein the drilling aid has been pivoted anticlockwise about the branch,

FIG. 7c shows a position comparable to FIG. 7a, wherein the drilling aid has been rotated clockwise through 180° about the longitudinal axis of the branch,

FIG. 7d shows a rotation position comparable to FIG. 7a, wherein this drilling aid is also shown to be rotatable about a rotation axis perpendicular to the forceps plane,

FIG. 8 shows a side view of the drilling aid from FIG. 7a, with the guide axis of the bore inclined with respect to the forceps plane, and

FIG. 9 shows a view comparable to FIG. 8 and depicting a third illustrative embodiment, in which the drilling aid is additionally designed to be movable along the branch on which it is mounted.

### DETAILED DESCRIPTION OF PREFERRED EMBODIMENTS

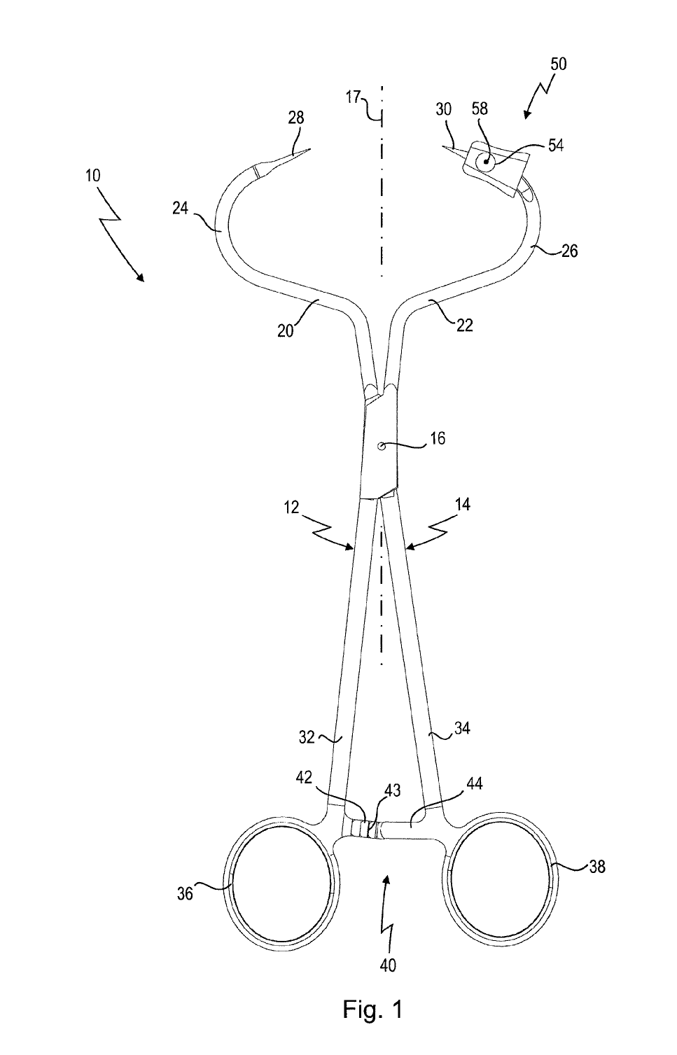

A first embodiment of repositioning forceps according to the invention is shown in FIGS. 1 to 6 and is designated in its entirety by reference number 10.

The repositioning forceps 10 has two forceps halves 12 and 14, which are connected to each other like scissors via a hinge 16 and are pivotable about this hinge 16. It will be seen from FIG. 1 that the forceps halves 12 and 14, seen along a central longitudinal axis 17 of the repositioning forceps 10, are more or less mirror-inverted.

The two forceps halves 12 and 14 can thus be pivoted in a forceps plane 18, which in FIG. 1 corresponds approximately to the plane of the drawing.

In the distal direction from the hinge 16, each forceps half 12, 14 has a branch 20, 22, respectively.

Each of the branches 20, 22 has a laterally curved portion 24, 26, respectively, extending in the forceps plane 18.

The two curved portions 24, 26 each merge in the distal direction into an approximately rectilinear needle-like spike 28, 30, respectively, the tips of which spikes 28, 30 are directed towards each other and can meet at the central longitudinal axis 17.

In the proximal direction from the hinge 16, each forceps half 12, 14 has a grip part 32, 34, respectively, which at the proximal end is in each case provided with a finger hole 36, 38, respectively.

A holding device 40 extends between the grip parts 32 and 34 in this area and consists of two ratch-like locking elements 42, 44 which are directed towards each other and overlap and which each protrude inwardly from the inside face of the grip part 32, 34, respectively.

In the view in FIG. 1, teeth 43 of the locking element 42 can be seen, which engage with corresponding teeth (not shown here) on the locking element 44.

In other words, the holding device 40 ensures that the repositioning forceps 10 remain in the position shown in FIG. 1, for example, when not moved.

However, the locking action is such that it can be overcome by the force of a human hand grasping the repositioning forceps 10, such that, by a corresponding movement, the repositioning forceps 10 in the view in FIG. 1 can be opened further or can also be correspondingly closed.

A drilling aid, designated in its entirety by reference number 50, is mounted on the branch 22. The drilling aid 50 is mounted at a distal end section of the branch 26 close to the needle tip 30.

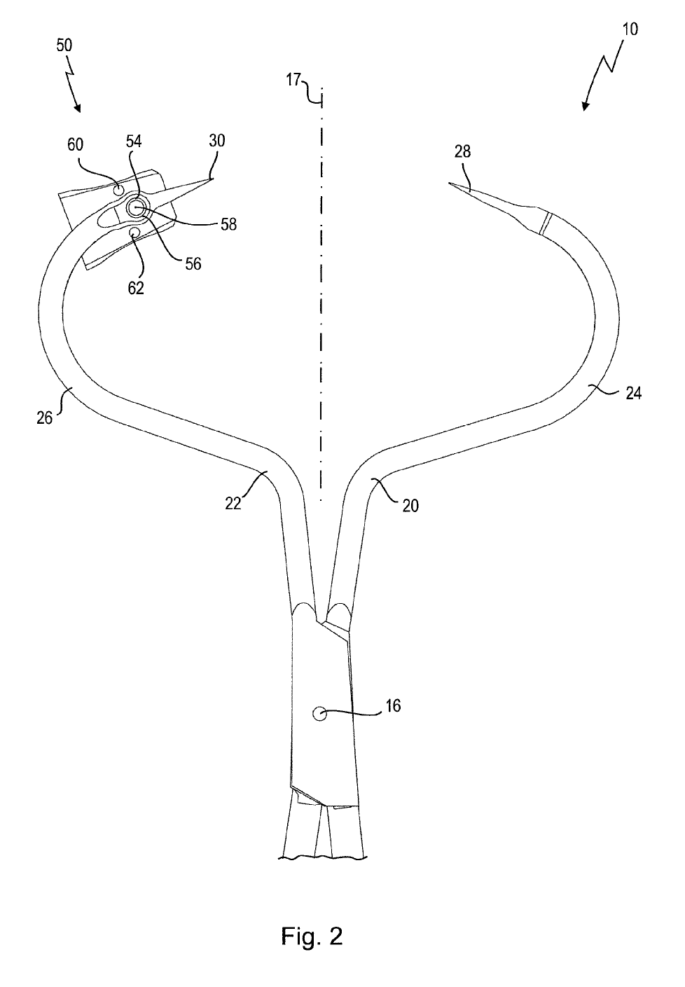

As can be seen in detail particularly from FIGS. 2 to 5, the drilling aid 50 consists of a more or less cuboid block 52.

A pin 54, which is mounted in an eyelet 56 in the branch 22, extends all the way through the block 52.

The drilling aid 50 can be rotated about the pin 54, the central longitudinal axis of the pin 54 representing a rotation axis 58 of the drilling aid 50. The rotation axis 58 extends perpendicular to the forceps plane 18.

The drilling aid 50 or the block 52 is in this way mounted laterally on the branch 22 via the pin 54, such that the drilling aid 50 is in principle rotatable through 360° about the rotation axis 58.

In the illustrative embodiment shown, however, this rotation movement about the rotation axis 58 is limited by means of two abutments 60 and 62 being provided on the block 52.

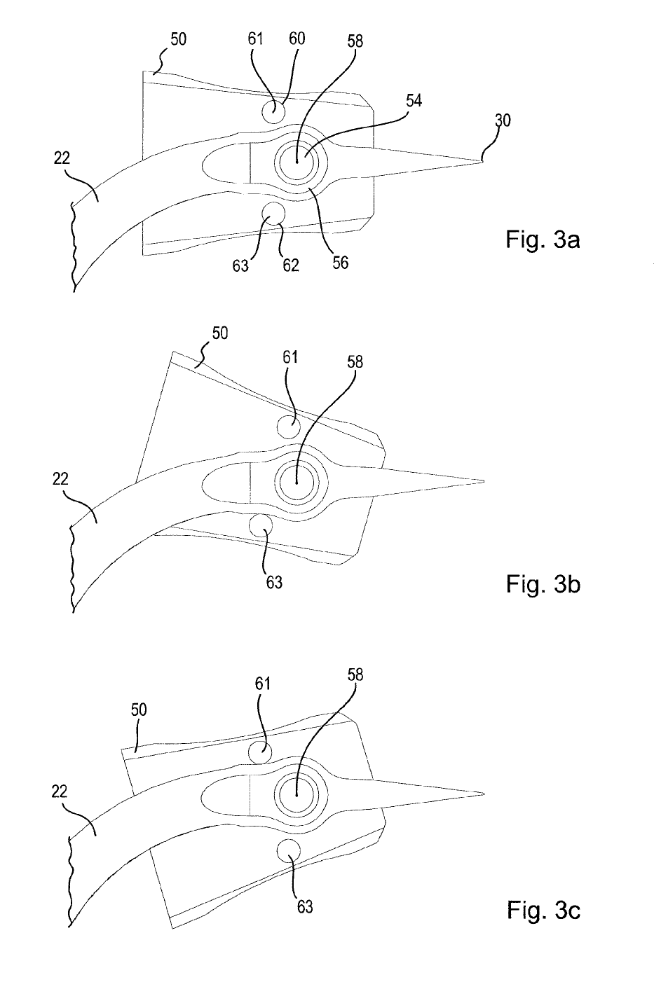

As can be seen in particular from FIGS. 3A to 3C and FIGS. 4 and 5, the abutments 60, 62 are designed as stubs 61 and 63 protruding towards the branch 22 from a flat outer face of the block 52. Looking along the branch 22 from the proximal to the distal end in the view in FIG. 3, the stub 61 extends to the left from the branch 22, and the stub 63 extends to the right from the branch 22.

The arrangement and the spacing of the abutments 60, 62 are such that, starting from a “central position” as shown in FIGS. 1, 2 and 3A, the drilling aid 50 can be pivoted clockwise or anticlockwise about the rotation axis 58 until one of the abutments 60, 62 strikes the outer face of the branch 22.

In the illustrative embodiment shown, the range of pivoting from the central position in FIG. 3A is in each case approximately 20°.

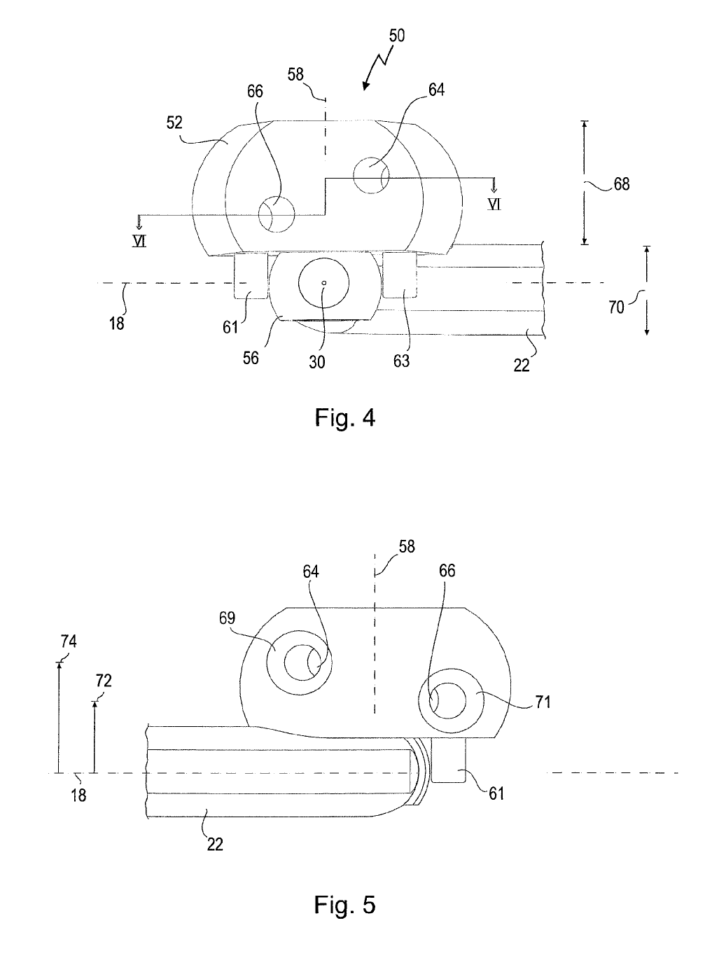

It will be seen from the views in FIGS. 4 to 6 that two guide channels 64 and 66 are present in the block 52 of the drilling aid 50. It is clear here that the two guide channels 64 and 66 extend completely through the block 52 in a direction designated as “longitudinal direction”. It will be seen that the guide channels 64 and 66 are formed at different heights, with the “upper” guide channel 64 at the height 74 and the “lower” guide channel 66 at the height 72.

The guide axes 65, 67 of the guide channels 64, 66, respectively, extend approximately parallel to the forceps plane 18, but at different heights, although, when viewed from proximal to distal, that is to say in the view in FIG. 5, they are directed toward each other and, seen in the direction of the longitudinal extent of the branch 22 at this point, run towards each other.

It will also be seen from the view in FIG. 5 that the guide channels 64 and 66 are provided with corresponding entry bevels 69 and 71, respectively, in order to facilitate the introduction of a drilling tool, for example a Kirschner wire or a screw drill.

It will also be seen from the view in FIG. 5 that the overall height 68 of the block 52 is only very slightly greater than the external diameter 70 of the branch 22, that is to say the block 52 is very compact in terms of height. It will also be seen that the branch 22 is flattened in the portion on which the block 52 is mounted. In this way the overall height is kept small in this area.

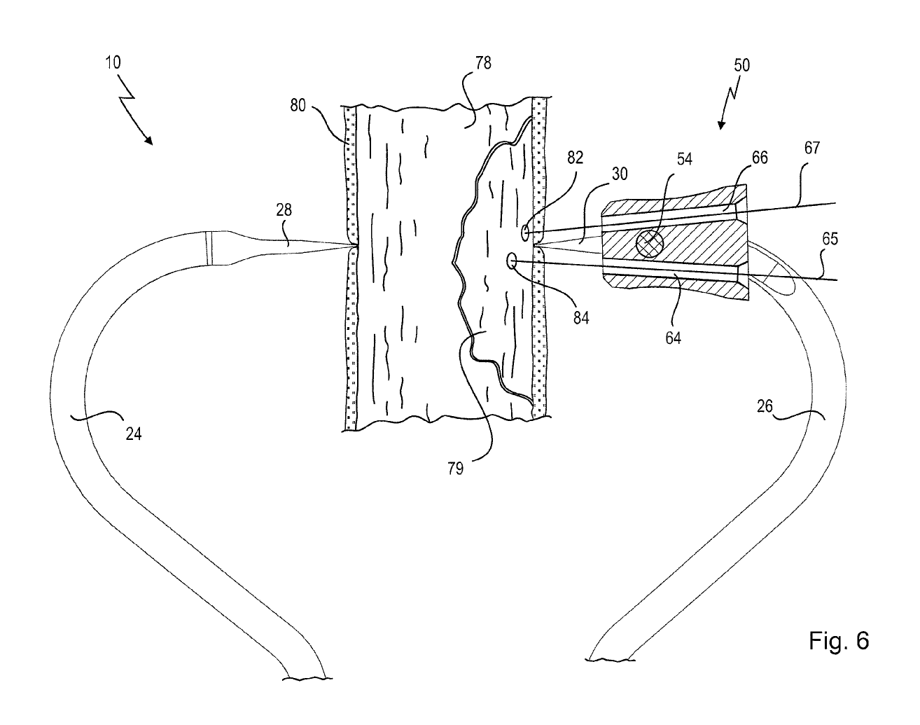

FIG. 6 shows the repositioning forceps 10 being manoeuvred in use such that a bone fragment 79 that has broken off from a bone 78 can be fixed back onto said bone.

It will be seen in FIG. 6 that the repositioning can be achieved by means of the needle-like spikes 28 and 30 being pushed from the outside through the skin 80 surrounding the bone 78, so as to place the broken-off bone fragment 79 in the correct position on the bone 78.

In this position, which is maintained by the aforementioned holding device 40, the operating surgeon can now orient the drilling aid 50 by rotating the latter in the desired manner around the pin 54.

By virtue of the fact that two guide channels 64 and 66 are formed in the block 52 of the drilling aid 50 and extend at different heights and are directed towards each other, two drill channels 82 and 84 can now be formed along the corresponding guide axes 65 and 67, through which drill channels 82 and 84 corresponding fixing screws or fixing wires can then be pushed. Thus, in one procedure, two such drill channels 82 and 84 can be produced which, after withdrawal of the drilling tool, are very easily accessible in order to fit the fixing screws or fixing wires.

FIG. 6 shows the drilling aid 50 approximately in the “central position” shown in FIG. 3A.

By suitable pivoting of the drilling aid 50 about the pin 54, it is also possible to produce the drill channels 82 and 84 at different locations, for example one channel further up in the view in FIG. 6, and another further down, depending on what is most favourable for fixing the broken-off bone fragment 79.

This demonstrates the easy and variable manoeuvring of the repositioning forceps 10 in an intervention.

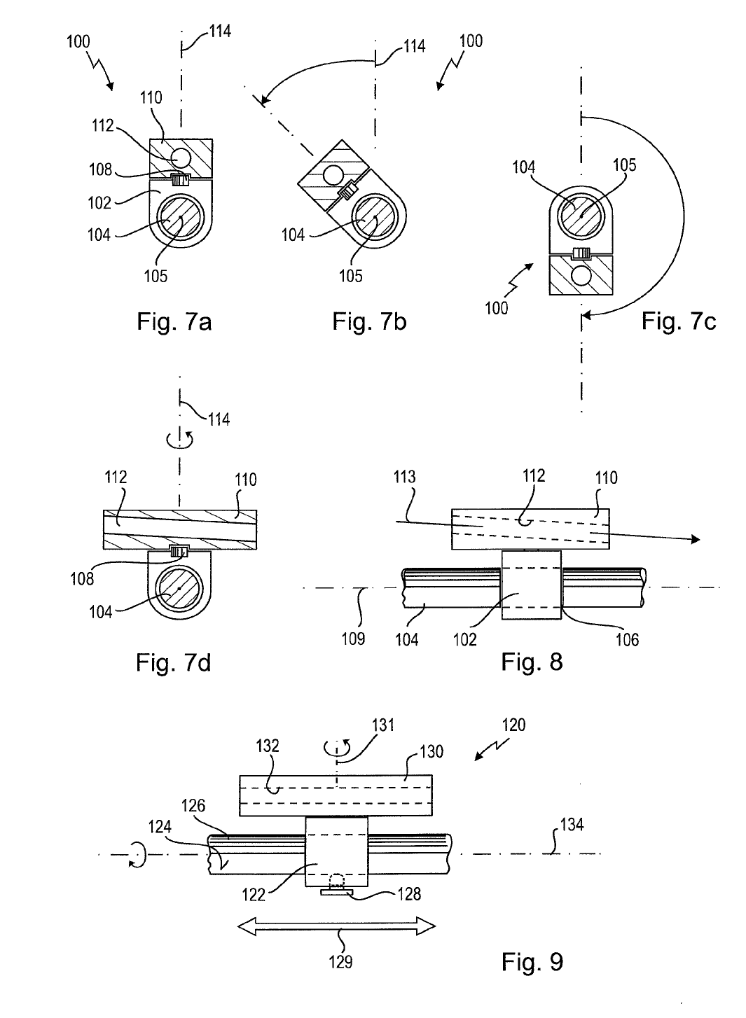

FIGS. 7a to 8 show a second embodiment of a repositioning forceps which, as regards the actual body of the forceps, is constructed in the same way as the repositioning forceps 10 shown in FIG. 1.

In this illustrative embodiment too, a drilling aid is provided, which is designated in its entirety here by reference number 100 and which is mounted on a branch 104, wherein the branch 104 corresponds to the branch 22 in FIG. 1.

From the cross section, which runs transversely with respect to a central longitudinal axis 105 of the branch 104, it will be seen that the drilling aid 100 has a sleeve 102, which here engages completely around the body of the branch 104.

The sleeve 102 sits in a circumferential groove 106 on the outside of the branch 104. At the upper end shown in FIG. 7a or the end remote from the branch 104, a rotary stub 108 is introduced on which a guide block 110 of the drilling aid 100 is mounted rotatably.

The central longitudinal axis of the rotary stub 108 forms a rotation axis 114 about which the guide block 110 is rotatable. It will also be seen from the sectional view in FIG. 7a that a guide channel 112 is formed in the guide block 110. From the sequence of FIGS. 7a, 7b and 7c, it is clear that the entire drilling aid 100 can be rotated in any desired manner about the central longitudinal axis 105 of the branch 104.

From the change from FIG. 7a to FIG. 7b, it will be seen that the drilling aid has been pivoted approximately through 30° anticlockwise. It will be seen from the view in FIG. 7c that the drilling aid 100 has been pivoted approximately through 180° about the central longitudinal axis 105. This pivoting or rotation movement is executed exactly by virtue of the fact that the sleeve 102 is fitted in and guided by a groove 160 cut into the outside of the branch 104. This pivotability about the longitudinal axis 105 of the branch 104 can be made suitably stiff, such that the drilling aid 100 remains in the respective rotation position. A securing device can also be provided, for example a radial locking screw.

FIGS. 7a to 7c show that the guide channel 112 extends in the direction of the central longitudinal axis 105 of the branch 104.

However, as has already been mentioned, the guide block 110 is rotatable about the rotation axis 114.

FIG. 7d shows how the guide block 110 is rotated through 90° about the rotation axis 114 in relation to the orientation in FIG. 7a.

From the views in FIG. 7d and FIG. 8, it will be seen that the guide axis 113 of the guide channel 112 extends at an inclination with respect to the forceps plane 109.

Depending on from which side the drilling tool is now pushed in through the guide channel 112, or in which rotation direction the latter is located, a bore can now be produced that is either inclined towards the forceps plane 109 or is directed away from the latter.

This once again demonstrates the versatility afforded to the operating surgeon for producing a suitable bore in the bone.

The inclined orientation of the guide axis 113 is only one illustrative embodiment; the guide channel 112 can also extend parallel to the forceps plane 109.

FIG. 9 shows a third illustrative embodiment of repositioning forceps, where once again the repositioning forceps as such are designed in the same way as the repositioning forceps 10 shown in FIG. 1.

In contrast to the second illustrative embodiment shown in FIGS. 7 and 8, the sleeve 122 in the third embodiment of the drilling aid 120 is designed such that it engages completely around and bears on the outer face 124 of the branch 126, which again corresponds to the branch 22 of the illustrative embodiment in FIG. 1.

A securing device 128, for example in the form of a radially extending locking screw, is present in the branch 126 in order to fix a defined rotation position of the sleeve 122 about the longitudinal axis 132 of the branch 126. The central longitudinal axis of the branch 126 lies in the forceps plane 134 of the repositioning forceps.

Here too, a guide block 130 is mounted rotatably on the sleeve via a rotary stub, such that the guide block 130 can be rotated about a rotation axis 131 perpendicular to the forceps plane 134.

Extending through the guide block 130 is a continuous guide channel 132, which is not inclined, that is to say its central longitudinal axis extends parallel to the forceps plane 134.

In this illustrative embodiment, the guide block 130 with the guide channel 132 is once again rotatable about a rotation axis 131 around the sleeve 122.

Moreover, the sleeve 122 itself, and therefore the complete drilling aid 120, is rotatable about the longitudinal axis of the branch 126, as has already been described above.

The third degree of freedom is now additionally provided, whereby the entire drilling aid 120 can also be moved along the branch 136, as is indicated by the double arrow 129, when the securing device is loosened.

In this embodiment, numerous degrees of freedom are now available to the operating surgeon, allowing the drilling aid to be brought to the optimum orientation for the particular application.

Of course, with the drilling aids shown in the second and third illustrative embodiments, it is also possible to produce several drill channels as are described in connection with FIG. 1.

1. A repositioning forceps for holding two bone parts together, which bone parts are to be connected, comprising
two scissors-like forceps halves,
a hinge connecting said two forceps halves like a scissors,
said two scissors-like forceps halves connected via said hinge extend and are movable in a forceps plane,
each of said two forceps halves has a distal section distally from said hinge and a proximal section proximally from said hinge,
each of said two forceps halves has a grip part at said proximal section,
each of said two forceps halves has a laterally outwardly curved branch in said distal section, said curved branches having distal ends directed towards each other in a closed position of said reposition forceps,
a drilling aid mounted at one of said curved branches, said drilling aid being rotable at least about a rotation axis extending perpendicular to said forceps plane, said drilling aid comprising a drilling tool guide having a guide axis, said guide axis extending, in a particular rotation position of said rotatable drilling aid along said branch having mounted said drilling aid, but can deviate from an extension along said branch when said drilling aid was turned about said rotation axis.

2. The repositioning forceps of claim 1, wherein said drilling aid is designed as a block through which at least one guide channel for guiding a drilling tool extends.

3. The repositioning forceps of claim 2, wherein at least two guide channels are formed in said block.

4. The repositioning forceps of claim 3, wherein guide axes of said guide channels extend at different heights but can nonetheless be oriented with respect to an area between said two branches.

5. The repositioning forceps of claim 3, wherein guide axes of said drilling tool guide extend non-parallel to each other but nonetheless be oriented with respect to an area between said two branches.

6. The repositioning forceps of claim 1, wherein said drilling aid being connected to said branch by a pin extending in said rotation axis.

7. The repositioning forceps of claim 1, wherein an extent of rotation of said drilling aid about said rotation axis is limited.

8. The repositioning forceps of claim 7, wherein abutments protrude from said drilling aid on both sides of said branch and each abutment limits a rotation about said rotation axis in opposite directions of rotation.

9. The repositioning forceps of claim 1, wherein said drilling aid is additionally designed to be rotatable about a longitudinal axis of said branch on which it is mounted.

10. The repositioning forceps of claim 1, wherein said drilling aid is additionally designed to be movable along said branch on which it is mounted.

11. The repositioning forceps of claim 1, wherein said two branches of said forceps halves are designed as needle-like spikes at its distal ends.

12. The repositioning forceps of claim 11, wherein said needle-like spikes are rectilinear.

13. The repositioning forceps of claim 12, wherein said branches, seen from a central longitudinal axis of said repositioning forceps, have laterally outwardly curved portions, of which said needle-like spikes are directed towards each other when said repositioning forceps is closed.

14. The repositioning forceps of claim 13, wherein said drilling aid is arranged near said needle-like spike of said branch.

15. The repositioning forceps of claim 1, wherein a holding device is present, which extends between said grip parts and by means of which holding device said grip parts can be held at defined distances.
